(19) United States
(12) Patent Application Publication
(10) Pub. No.: US 2026/0033757 A1
(43) Pub. Date: Feb. 5, 2026

(54) TRANSCUTANEOUS CO2 SENSOR AND USES THEREOF

(71) Applicant: BIOSENCY, Saint-Grégoire (FR)

(72) Inventors: Yann LE GUILLOU, Saint-Grégoire (FR); Emmanuel DERVIEUX, Saint-Grégoire (FR)

(73) Assignee: BIOSENCY, Saint-Grégoire (FR)

(21) Appl. No.: 18/793,167

(22) Filed: Aug. 2, 2024

(51) Int. Cl. A61B 5/1455; A61B 5/00; A61K 49/00

(52) U.S. Cl. CPC A61B 5/14556; A61B 5/14552; A61B 5/6833; A61K 49/0054

(57) ABSTRACT

A sensor for transcutaneous determination of arterial CO₂ partial pressure using luminescence and a method for transcutaneous determination of arterial CO₂ partial pressure using luminescence.

### FIELD

The present invention relates to sensors for transcutaneous determination of blood gases pressure, in particular carbon dioxide, and method for said determination.

### BACKGROUND

Carbon dioxide (CO₂) diffusion through the skin enables the non-invasive measurement of transcutaneous partial CO₂ pressure (tcpCO₂), a physiological value that correlates with arterial partial CO₂ pressure (paCO₂) which is of major clinical significance. Indeed, the accurate determination of carbon dioxide arterial blood content is of crucial importance in medical care since it gives circulatory as well as ventilatory clues on the state of a patient.

Commonly used methods to measure paCO₂ are arterial blood sampling, airway capnometry and transcutaneous capnometry. Arterial blood sampling is the gold standard for paCO₂ measurement. However, it is invasive, can be both painful and risked, needs expensive blood gas analyzer, and the blood samples must be analyzed quickly upon collection. To circumvent these flaws, two capnometry methods were developed. First, airway capnometry, also known as capnography, was developed. However, it is also invasive, since it requires the patient to wear a mask or undergo endotracheal intubation, and is not reliable in case of petCO₂/paCO₂ mismatch caused by an increase in physiological dead space. Second, transcutaneous capnometry, derived from the Stow-Severinghaus electrode, was developed and has been used for a long time in the clinical practice. Its drawbacks are mainly the need to heat the skin of the patient between 37° C. and 44° C., the need for frequent recalibrations of the sensor, the high price of transcutaneous CO₂ monitors and the bulkiness of such devices. These monitoring devices cannot be used daily by patients alone because of the need for skilled personnel to perform equipment re-calibrations. Furthermore, such monitoring devices cannot be used for more than 8 hours on the same skin site due to the risk of skin burns induced by heating.

Consequently, there is a need for non-invasive, long-term, compact, calibration-free, transcutaneous CO₂ sensing.

tcpCO₂ measurements may be performed within a reasonable response time only if using a compact sensor, which exhibits a volume to surface ratio as small as possible, such a constraint drastically reduces the number of techniques usable for transcutaneous CO₂ monitoring.

Thus, a new generation of transcutaneous CO₂ is presented herein relying on luminescence properties of luminophores to determine paCO₂, with the advantages of having a reduced maintenance cost, smaller drift, and the possibility to replace only the CO₂-sensitive part of the sensor, without changing the optical part when the sensor ages.

### SUMMARY

A sensor for transcutaneous determination of arterial CO₂ partial pressure using luminescence, said sensor comprising:
a patch assembly comprising:
a sealing layer configured to ensure a contact between skin and the sensor;
a CO₂-permeable polymer layer configured to allow diffusion of CO₂ from the skin in the patch assembly;
a CO₂-sensitive luminescent layer comprising a pH sensitive luminophore and a non-pH sensitive luminophore;
a CO₂-impermeable polymer layer embedding the CO₂-sensitive luminescent layer;
a sensing head comprising:
a substrate;
at least one light emitting diode configured to emit light to excite the luminophores in the CO₂-sensitive luminescent layer;
at least one photodiode configured to detect light emitted by the luminophores in the CO₂-sensitive luminescent layer;
a housing partially or totally encapsulating the substate, the at least one light emitting diode and the at least one photodiode;
wherein the arterial CO₂ partial pressure is determined by comparison of at least one luminescence property of the pH sensitive luminophore and the non-pH sensitive luminophore;
wherein the mass ratio between the pH sensitive luminophore and the non-pH sensitive luminophore in the CO₂-sensitive luminescent layer is ranging from 10% and 90%.

According to one advantageous aspect of the invention, the loading charge of the pH sensitive luminophore in the CO₂-sensitive luminescent layer is at least 0.5%, said loading charge being the mass ratio between the mass of luminophore in the CO₂-sensitive luminescent layer and the mass of said CO₂-sensitive luminescent layer.

According to one advantageous aspect of the invention, the loading charge of the non-pH sensitive luminophore in the CO₂-sensitive luminescent layer is at least 0.5%, said loading charge being the mass ratio between the mass of luminophore in the CO₂-sensitive luminescent layer and the mass of said CO₂-sensitive luminescent layer.

According to one advantageous aspect of the invention, the pH sensitive luminophore is selected among 1-hydroxy-pyrene-3,6,8-trisulfonate, (2′, 7′-Bis-(2-Carboxyethyl)-5-Carboxyfluorescein), seminaphtharhodafluorescein, 4,4-difluoro-5,7-dimethyl-4-bora-3a, 4a-diaza-s-indacene-3-propionic acid, other fluorescein derivatives, bromothymol blue, methyl red, litmus, cresol blue, bromocresol purple, chlorophenol red, phenol red, naphtolphtalein, phenolphthalein, cresolphtalein, nitrophenol, azolitmin, neutral red, rosolic acid, tropeolin OOO (1 and 2), or a mixture thereof. These luminophores are advantageously pH-sensitive and have a short luminescence lifetime.

According to one advantageous aspect of the invention, non-pH sensitive luminophore is selected among tris(4,7-diphenyl-1,10 phenanthroline) ruthenium (II) dichloride (Ru-dpp), tris(1,10 phenanthroline) ruthenium (II) dichloride (Ru-pn), tris (2,2′-bipyridyl) ruthenium (II) dichlo-ride (Ru-bpy), pyrene, platinum octaethylporphyrin (ketone) (PtOEP (K)), palladium tetraphenyl tetrabenzoporphine (PdTPTBP), platinium tetraphenyl tetrabenzoporphine (PtTPTBP), Ir(Cn)2(acac), Ir(Cs)2(acac), cyclometalated iridium (III), coumarin complexes, or a mixture thereof. These luminophores are advantageously not pH sensitive and have a long luminescence lifetime.

According to one advantageous aspect of the invention, the CO₂-sensitive luminescent layer comprises:
a first pH sensitive layer comprising the pH sensitive luminophore dispersed in a first polymer matrix;
a second non-pH sensitive layer comprising the non-pH sensitive luminophore dispersed in a second polymer matrix and being deposited on top of the first layer.

According to one advantageous aspect of the invention, the CO₂-sensitive luminescent layer comprises a mixture of the pH sensitive luminophore and the non-pH sensitive luminophore in a polymer matrix.

According to one advantageous aspect of the invention, the CO₂-sensitive luminescent layer comprises:
a polymer matrix;
the pH sensitive luminophore dispersed in the polymer matrix; and
polymer beads comprising the non-pH sensitive luminophore in a polymer, said polymer beads being dispersed in the polymer matrix.

According to one advantageous aspect of the invention, the polymer matrix comprises ethyl cellulose, methyl cellulose, hydroxyethyl cellulose, hydroxymethyl cellulose, sodium carboxymethyl cellulose, hydroxyethyl methacrylate, hydroxypropyl methylcellulose, starches, gelatins, polyacrylonitrile (PAN), polytetrafluoroethylene (PTFE), polyethylene (PET), polystyrene (PS), poly (butylene adipate) polyester-based polyurethane, poly(methyl methacrylate) (PMMA), polyvinyl fluoride (PVF), poly(vinylidene chloride-co-vinyl chloride) (PVCD), BTDA-p,p′-DAS, poly (p-phenylene terephthalamide) (PPTA), poly(isobutyl methacrylate) (polyIBM), ethylene-vinyl alcohol copolymer (EVOH), polyvinyl alcohol (PVA), silicone, ormosil glass, or a mixture thereof.

According to one advantageous aspect of the invention, the arterial CO₂ partial pressure is determined using Förster Resonance Energy Transfer, dual-lifetime referencing, Inner Filter Effect, or ratiometric probing between the pH sensitive luminophore and the non-pH sensitive luminophore. These determination methods have advantageously a rapid time response allowing real-time monitoring of CO₂ concentrations.

According to one advantageous aspect of the invention, the CO₂-sensitive luminescent layer has a thickness ranging from 1 μm to 100 μm, preferably ranging from 10 μm to 15 μm. Advantageously, as the exhalation rate of CO₂ from human skin is low, a thin CO₂-sensitive luminescent layer is equilibrated quickly with the skin CO₂ content.

According to one advantageous aspect of the invention, the patch assembly has a thickness ranging from 20 μm to 500 μm, preferably ranging from 150 μm to 350 μm.

According to one advantageous aspect of the invention, the CO₂-permeable polymer layer comprises polytetrafluoroethylene, poly (dimethylsiloxane), hyflon, or a mixture thereof. Advantageously, these materials allow CO₂ diffusion from skin to the CO₂-sensitive luminescent layer while ensuring a protection against humidity loss and acidic vapors or sweat poisoning.

The invention also relates to a method for transcutaneous determination of arterial CO₂ partial pressure using luminescence comprising:
providing a sensor according to this disclosure;
placing said sensor on skin of a subject;
exciting the luminophores in the CO₂-sensitive luminescent layer with the at least one light emitting diode;
detecting light emitted by said luminophores with the at least one photodiode;
comparing luminescence properties of the pH sensitive luminophore and the non-pH sensitive luminophore;
determining arterial CO₂ partial pressure based on said comparison.

According to one advantageous aspect of the invention, the arterial CO₂ partial pressure is determined using Förster Resonance Energy Transfer, dual-lifetime referencing, Inner Filter Effect, or ratiometric probing between the pH sensitive luminophore and the non-pH sensitive luminophore.

The invention also relates to a method for fabricating a sensor according to this disclosure comprising:
assembling the patch assembly of the sensor;
assembling the sensing head of the sensor; and
coupling the patch assembly with the sensing head.

### Definitions

In the present invention, the following terms have the following meanings:

“Luminescence” refers to the emission of light by a substance/material that has not been heated. It occurs when a substance/material absorbs energy and then re-emits it as light. Common types of luminescence include fluorescence, phosphorescence, chemiluminescence, and bioluminescence.

“Luminophore” refers to a substance/material capable of luminescence. It includes fluorophores or phosphors.

“NIR” refers to light of wavelength in the range from 780 nm to 1400 nm.

“Optically transparent” refers to a material that absorbs less than 30%, 20%, 10%, 5%, 1%, or 0.5% of light at a determined wavelengths.

“pH-sensitive luminophore” refers to a luminophore that undergoes structural modification depending on the pH. It consists in an acid-base couple RH/R⁻. When the pH of its surrounding medium is low, the protonated specie RH is predominant, while in a more basic medium, the anionic specie R⁻ is. The optical probing is made possible by the fact that RH and R⁻ exhibit different optical properties.

“UV” refers to light of wavelength in the range from 10 nm to 380 nm. In particular, UVA refers to the sub-range of UV from 315 nm to 380 nm.

“UVA-Visible-NIR” refers to light of wavelength in the range from 315 nm to 1400 nm.

“Visible” refers to light of wavelength in the range from 380 nm to 780 nm.

### DETAILED DESCRIPTION

### Sensor for Transcutaneous Determination of Arterial CO₂ Partial Pressure

This invention relates to a sensor for transcutaneous determination of arterial CO₂ partial pressure using luminescence.

Said sensor comprises:
a patch assembly comprising:
   a sealing layer configured to ensure a contact between skin and the sensor;
   a CO₂-permeable polymer layer configured to allow diffusion of CO₂ from the skin in the patch assembly;
   a CO₂-sensitive luminescent layer comprising a pH sensitive luminophore and a non-pH sensitive luminophore (or pH insensitive luminophore);
   a CO₂-impermeable polymer layer embedding the CO₂-sensitive luminescent layer;
   a sensing head comprising:
   a substrate;
   at least one light emitting diode (LED) configured to emit light to excite the luminophores in the CO₂-sensitive luminescent layer;
   at least one photodiode configured to detect light emitted by the luminophores in the CO₂-sensitive luminescent layer;
   a housing partially or totally encapsulating the substate, the at least one light emitting diode and the at least one photodiode.

The arterial CO₂ partial pressure may be determined by comparison of at least one luminescence property of the pH sensitive luminophore and the non-pH sensitive luminophore.

Comparison of luminescence properties means herein comparison of luminescence intensity, and/or luminescence lifetime.

The mass ratio between the pH sensitive luminophore and the non-pH sensitive luminophore in the CO₂-sensitive luminescent layer may be ranging from 10% to 90%, preferably from 25% to 75%, more preferably from 30% to 70%.

The sensor is placed against the skin so that the sealing layer of the patch assembly contact the skin in a gas-tight manner and CO₂ is allowed to diffuse through the skin inside the sensor Upon diffusion from the skin into the first three layers of the patch assembly, in particular the CO₂-sensitive luminescent layer, diffused CO₂ modifies the pH of said luminescent layer. At least one luminescence properties of the pH sensitive luminophore is modified due to pH change while the luminescence properties of the non-pH sensitive luminophore act as a reference signal because they remain stable and unaffected by changes in pH or CO₂ levels. This stability provides a constant reference point against which the changes in the pH sensitive luminophore luminescence can be measured. In particular, when the pH in the CO₂-sensitive luminescent layer is low, the protonated specie of the pH sensitive luminophore RH is predominant, while in a more basic medium, the anionic specie R⁻ is predominant, or the opposite. The optical probing is made possible by the fact that RH and R⁻ exhibit different optical properties: intensity, lifetime, or others.

The luminescence of both luminophores is then probed using the sensing head: upon excitation by the LED of the sensing head, both luminophores exhibit distinct luminescence properties, for example they emit light at different wavelengths or have different lifetimes in response to this excitation. Then, the photodiode collects the emitted light from both luminophores, and the comparison of at least one luminescent property of both luminophores allow to determine paCO₂.

The chosen sensing principle is equilibrium state sensing (as opposed to rate-based sensing): the sensor is placed against the skin, and CO₂ is allowed to diffuse through the skin inside the sensor until an equilibrium is reached between the sub-cutaneous tissues and the sensor. The pCO₂ inside the sensor at the end of the equilibration time is equal to the subject's tcpCO₂, and the response time of the sensor is solely function of the sensor height (or thickness). A small thickness is required to achieve small time response (inferior to 10 min). This could not be achieved with traditional CO₂ sensor.

Each layer of the patch assembly has its sole purpose. First, the sealing layer ensures that the sensor remains snug against the skin with no air gap. Second, the CO₂ permeable layer ensures that CO₂ can diffuse from the skin to the CO₂-sensitive luminescent layer. Third, the CO₂-sensitive luminescent layer allows the determination of pCO₂ based on the comparison of optical properties of both luminophores. As those first three layers are thin, the equilibration time will be reduced compared to bigger traditional CO₂ monitors. Fourth, the upper CO₂ impermeable layer guarantees that no CO₂ leakage occurs from the CO₂-sensitive luminescent layer to the ambient air, enhancing the time response and accuracy of the sensor. It also protects the patch assembly from physical aggressions

The sensor has several advantages over traditional CO₂ monitors:
accuracy and stability;

Using a non-pH-sensitive reference luminophore helps to improve the accuracy and stability of CO₂ measurements, as it accounts for potential variability in the sensor environment or signal strength;
non-invasive monitoring;

This sensor allows for non-invasive, continuous monitoring of CO₂ levels through the skin, making it useful in medical applications such as patient monitoring;
reduced maintenance cost;
smaller drift; and
the possibility to replace only the CO₂-sensitive part of the sensor, without changing the optical part when the sensor ages;

When the patch assembly wears out, or when the patient wants to remove it after a few days for hygiene reasons for instance, they can safely throw the patch assembly away while keeping the sensing head for other measurements. New patch assemblies can be provided in blister-packaged form and the patch assembly itself is a consumable. The sensing head, on its part, does not wear out. It may be used with multiple patch assemblies on the same patient, or even on multiple patients, given it is disinfected between one and the next.

Such technique would allow for long-term telemonitoring out of the hospital, while minimizing costs and patient discomfort.

The arterial CO₂ partial pressure may be determined using Förster Resonance Energy Transfer, dual-lifetime referencing, Inner Filter Effect, or ratiometric probing between the pH sensitive luminophore and the non-pH sensitive luminophore. These determination methods have advantageously a rapid time response allowing real-time monitoring of CO₂ concentrations.

Dual-Lifetime Referencing comprises time-based Dual-Lifetime Referencing (t-DLR) and frequency-based Dual-Lifetime Referencing (f-DLR).

In a preferred configuration, the arterial CO₂ partial pressure is determined by time-based Dual-Lifetime Referencing (t-DLR). t-DLR is a technique used to measure the luminescence lifetimes of both luminophores. By comparing the lifetime of the pH-sensitive luminophore to that of the non-pH-sensitive luminophore, it is possible to accurately determine the CO₂ pressure. This comparison helps to compensate for any potential fluctuations in signal strength, environmental changes, or sensor movement, leading to more reliable measurements. In other words, when the LED emits light to excite both luminophores, the rise of luminescence intensity of the pH sensitive luminophore (I_{FLU}) is almost instantaneous compared to that of the non-pH sensitive luminophore (I_{REF}). Similarly, when the LED stops emitting light, the decay in luminescence intensity of the pH sensitive luminophore is also instantaneous, leaving only the slow decay of the non-pH sensitive luminophore. pCO₂ can then be directly calculated from the IFLU/IREF ratio.

In a preferred configuration, the arterial CO₂ partial pressure is determined by frequency-based Dual-Lifetime Referencing (f-DLR). f-DLR is a technique used to measure the concentration ratio of both luminophores. By illuminating the two luminophores with a sinusoidal light excitation signal and measuring the phase shift between the re-emitted signal and the excitation signal, the ratio of the concentrations of the reference luminophore and the pH-sensitive fluorophore can be retrieved. From this ratio, it is possible to accurately determine the CO₂ pressure. This comparison helps to compensate for any potential fluctuations in signal strength, environmental changes, or sensor movement, leading to more reliable measurements.

In a preferred configuration, the arterial CO₂ partial pressure is determined using Förster Resonance Energy Transfer (FRET). FRET is a distance-dependent interaction between two light-sensitive molecules (luminophores): a donor and an acceptor. It involves the non-radiative transfer of energy from the excited state of the donor luminophore to the acceptor luminophore, which then emits light. FRET will take place between the non-pH sensitive luminophore L, and the anionic form of the pH sensitive luminophore R⁻, according to:

$${L}^{*} + {R}^{-} {\to }^{FRET} L + {R}^{- *} \qquad ( 1 )$$

where R⁻ returns to its de-excited state through non-radiative thermal relaxation. The more CO₂ present, the lower the pH and the less R⁻ available for FRET, leading to a higher luminescence intensity. At the opposite, the less CO₂ present, the higher the pH. More pH sensitive luminophore will then be present in its R⁻ form, effectively quenching the non-pH sensitive luminophore L luminescence via FRET. A measurement of luminescence quenching by FRET can thus yield pCO₂ value inside the sensor.

In an alternate configuration, the arterial CO₂ partial pressure is determined by Inner Filter Effect. In this case, the pH sensitive luminophore is colorimetric pH sensitive. The luminophores are chosen such that the absorption spectrum of at least one of the two forms (protonated or anionic) of the pH sensitive luminophore overlaps with the emission spectrum of the non-pH sensitive luminophore. Under this condition, the luminescence of the non-pH sensitive luminophore will be more or less quenched by the pH sensitive luminophore as a function of the surrounding pH. The pH sensitive luminophore thus acts as a filter, absorbing a fraction of the light emitted by the non-pH sensitive luminophore.

The loading charge of the pH sensitive luminophore in the CO₂-sensitive luminescent layer may be at least 0.5%, said loading charge being the mass ratio between the mass of luminophore in the CO₂-sensitive luminescent layer and the mass of said CO₂-sensitive luminescent layer. Said loading charge may be at least 1%, 5% or 10%.

The loading charge of the non-pH sensitive luminophore in the CO₂-sensitive luminescent layer may be at least 0.5%, said loading charge being the mass ratio between the mass of luminophore in the CO₂-sensitive luminescent layer and the mass of said CO₂-sensitive luminescent layer. Said loading charge may be at least 1%, 5% or 10%.

In a first configuration, the CO₂-sensitive luminescent layer may comprise:
a first pH sensitive layer comprising the pH sensitive luminophore dispersed in a first polymer matrix;
a second non-pH sensitive layer comprising the non-pH sensitive luminophore dispersed in a second polymer matrix and being deposited on top of the first layer.

The pH sensitive luminophore and the non-pH sensitive luminophore do not need to be in close contact with each other to allow the determination of pCO₂. Having two layers, each comprising one of the luminophores, advantageously allows to select the polymer matrix for each luminophore. Indeed, the use of two luminophores with very distinct purposes may call for two corresponding polymers with very distinct properties. For example, the first polymer matrix must have a low O₂ permeability (low gaseous solubility and diffusivity) to act as an oxygen barrier in order to encapsulate the non-pH sensitive luminophore. At the opposite, the second polymer matrix must be highly hydrophilic/hygroscopic, and facilitate the diffusion of CO₂ in order to allow for a good acid-basic conversion of the pH sensitive luminophore and detection of the latter gas.

The first polymer matrix and the second polymer matrix may be the same polymer or distinct polymers.

The first polymer matrix is preferably hydrophilic to advantageously facilitate the diffusion of CO₂ in order to allow for a good acid-basic conversion of the pH sensitive luminophore and detection of the latter gas.

The first pH sensitive layer and the second non-pH sensitive layer may have the same or distinct thickness, preferably ranging from 1 μm to 50 μm, preferably ranging from 5 μm to 20 μm.

In this configuration, the CO₂-sensitive luminescent layer may further comprise a third inert polymer layer located between the first and second layers.

In a second alternate configuration, the CO₂-sensitive luminescent layer may comprise a mixture of the pH sensitive luminophore and the non-pH sensitive luminophore in the same polymer matrix. Advantageously, this configuration is easier to manufacture: a single batch of polymer matrix containing both luminophores is prepared, rather than having to make two separate layers.

In a third alternate configuration, the CO₂-sensitive luminescent layer may comprise:
a polymer matrix;
the pH sensitive luminophore dispersed in the polymer matrix; and
polymer beads comprising the non-pH sensitive luminophore in a polymer, said polymer beads being dispersed in the polymer matrix.

Advantageously, this configuration is easy to manufacture as the polymer beads may be prepared in advance and stored, so that a single batch of polymer matrix, polymer beads and pH-sensitive luminophore is prepared and coated on the previous layer (CO₂-impermeable layer). This also means that only one layer needs to be made, and compared with the previous configuration, it potentially allows the reference luminophore to be better encapsulated.

The pH sensitive luminophore may have a shorter luminescence lifetime than the non-pH sensitive luminophore. This is particularly advantageous for f-DLR or t-DLR determination: the non-pH sensitive luminophore is chosen so that its luminescence lifetime is much longer than that of the pH sensitive luminophore, and so that its luminescence intensity remains constant with respect to the analyte, CO₂ in our case. The pH sensitive luminophore on its part, is chosen so that its luminescence time is much shorter than that of non-pH sensitive luminophore, and so that its luminescence intensity depends on the analyte. By illuminating both luminophores with a sinusoidal excitation signal of adequate frequency, the phase of the re-emitted light will depend on the analyte concentration.

The pH sensitive luminophore may be short-lived, i.e. having a luminescence lifetime ranging from 1 ns to 100 ns, preferably from 1 ns to 50 ns, more preferably from 1 ns to 25 ns.

The non-pH sensitive luminophore may be long-lived, i.e. having a luminescence lifetime ranging from 0.2 μs to 100 μs, preferably from 1 μs to 50 μs, more preferably from 2 μs to 10 μs.

The non-pH sensitive luminophore and the pH sensitive luminophore present a luminescence lifetime ratio of at least 3, preferably at least 10, more preferably at least 100.

The non-pH sensitive luminophore and the pH sensitive luminophore present a luminescence lifetime ratio ranging from 3 to 500.

The excitation spectra of the luminophores may overlap. This advantageously allows both luminophores to be excited by the same LED at the same wavelength. In particular, both luminophores may present an absorption peak centered around 450 nm (from 380 nm to 500 nm, preferably from 425 nm to 475 nm).

Both luminophores may have an emission peak ranging from 500 nm to 900 nm, preferably from 500 nm to 650 nm.

The pH sensitive luminophore may have a pKA ranging from 5 to 9, preferably from 7 to 10.

The pH sensitive luminophore may be colorimetric pH sensitive, i.e. exhibiting distinct absorption spectra depending on the surrounding pH, or luminescent pH sensitive, i.e. exhibiting distinct emission spectra depending on the surrounding pH.

The pH sensitive luminophore may be selected among 1-hydroxy-pyrene-3,6,8-trisulfonate (C₁₆H₇Na₃O₁₀S₃) HPTS, (2′,7′-Bis-(2-Carboxyethyl)-5-Carboxyfluorescein) (C₂₁H₁₂O₇), seminaphtharhodafluorescein (C₂₄H₁₅NO₄), 4,4-difluoro-5,7-dimethyl-4-bora-3a,4a-diaza-s-indacene-3-propionic acid (C₁₄H₁₅BF₂N₂O₂), other fluorescein derivatives, or a mixture thereof. These luminophores are advantageously pH-sensitive and have a short luminescence lifetime.

Preferably, the pH sensitive luminophore is HPTS (1-hydroxy-pyrene-3,6,8-trisulfonate (C₁₆H₇Na₃O₁₀S₃)).

If the pH-sensitive luminophore is absorbing only, such as that which may be used in case of Inner Filter Effect Sensing, it can be selected among bromothymol blue, methyl red, litmus, cresol blue, bromocresol purple, chlorophenol red, phenol red, naphtolphtalein, phenolphthalein, cresolphtalein, nitrophenol, azolitmin, neutral red, rosolic acid, tropeolin OOO (1 and 2), or a mixture thereof.

The non-pH sensitive luminophore may be selected among tris(4,7-diphenyl-1,10 phenanthroline) ruthenium (II) dichloride (Ru-dpp), tris(1,10 phenanthroline) ruthenium (II) dichloride (Ru-pn), tris(2,2′-bipyridyl) ruthenium (II) dichlo-ride (Ru-bpy), pyrene, platinum octaethylporphyrin (ketone) (PtOEP(K)), palladium tetraphenyl tetrabenzoporphine (PdTPTBP), platinium tetraphenyl tetrabenzoporphine (PtTPTBP), Ir(Cn)₂(acac), Ir(Cs)₂(acac), cyclometalated iridium (III), coumarin complexes, or a mixture thereof. These luminophores are advantageously not pH sensitive and have a long luminescence lifetime.

Preferably, the non-pH sensitive luminophore is Ru-dpp (tris(4,7-diphenyl-1,10 phenanthroline) ruthenium (II) dichloride).

In a preferred configuration, the pH sensitive luminophore is HPTS and the non-pH sensitive luminophore is Ru-dpp.

For illustrative purposes, in the preferred configuration wherein non-pH sensitive luminophore R is Ru-dpp and pH sensitive luminophore P is HPTS, the equilibrium equation for reaction with CO₂ is:

$${( {H}_{2} O )}_{x} \cdot {Q}^{+} {P}^{-} + {CO}_{2} ⇌ {( {H}_{2} O )}_{x - 1} \cdot {Q}^{+} PH + {HCO}_{3}^{-} \qquad ( 2 )$$

wherein Q⁺ is the quaternary ammonium cation used in the luminescent layer synthesis. Using conditions of neutrality and mass conservation can lead to a relation of the form:

$${p CO}_{2} = f ( {[ P ]}_{TOT} , [ {Q}^{+} ] , [ {P}^{-} ] ) \qquad ( 3 )$$

wherein [P]_{TOT} is the total HPTS concentration (both anionic and acid forms), and f is a rational function with respect to [P⁻], hence the link between pCO₂ and [P⁻].

The polymer matrix is preferably optically transparent in the range of wavelengths emitted by the LED and the luminophores.

The polymer matrix into which the pH-sensitive luminophore will be dispersed may comprise ethyl cellulose, methyl cellulose, hydroxyethyl cellulose, hydroxymethyl cellulose, sodium carboxymethyl cellulose, hydroxyethyl methacrylate, hydroxypropyl methylcellulose, starches, gelatins or a mixture thereof. Advantageously, these polymers are highly hydrophilic, which will provide water molecules for the hydration of CO₂ and the acid-base conversion of the pH-sensitive luminophore between its anionic and protonated forms.

The polymer matrix to which the non-pH-sensitive luminophore will be dispersed may comprise may comprise polyacrylonitrile (PAN), polytetrafluoroethylene (PTFE), polyethylene (PET), polystyrene (PS), poly(butylene adipate) polyester-based polyurethane, poly(methyl methacrylate) (PMMA), polyvinyl fluoride (PVF), poly(vinylidene chloride-co-vinyl chloride) (PVCD), BTDA-p,p′-DAS, poly (p-phenylene terephthalamide) (PPTA), poly(isobutyl methacrylate) (polyIBM), ethylene-vinyl alcohol copolymer (EVOH), polyvinyl alcohol (PVA), silicone, ormosil glass, or a mixture thereof. Advantageously, these polymers show low oxygen permeability, which is essential to prevent the non-pH-sensitive luminophore from being quenched or photobleach.

The polymer matrix may comprise ethyl cellulose, methyl cellulose, hydroxyethyl cellulose, hydroxymethyl cellulose, sodium carboxymethyl cellulose, hydroxyethyl methacrylate, hydroxypropyl methylcellulose, starches, gelatins, polyacrylonitrile (PAN), polytetrafluoroethylene (PTFE), polyethylene (PET), polystyrene (PS), poly(butylene adipate) polyester-based polyurethane, poly(methyl methacrylate) (PMMA), polyvinyl fluoride (PVF), poly(vinylidene chloride-co-vinyl chloride) (PVCD), BTDA-p,p′-DAS, poly (p-phenylene terephthalamide) (PPTA), poly(isobutyl methacrylate) (polyIBM), ethylene-vinyl alcohol copolymer (EVOH), polyvinyl alcohol (PVA), silicone, ormosil glass, or a mixture thereof.

The CO₂-sensitive luminescent layer may further comprise a phase transfer agent and/or a plasticizer. Advantageously, the role of the plasticiser is to facilitate the diffusion of CO₂ inside the luminescent layer, while the phase transfer agent provides an aqueous-like environment for the luminophore and CO₂.

Examples of phase transfer agent include, without being limited to, a quaternary ammonium anion, such as tetraoctyl ammonium hydroxide (TOAH), tetramethyl ammonium hydroxide (TMAH), cetyltrimethyl ammonium hydroxide (CTAH), or tetraoctyl ammonium bromide (TOAB). The phase transfer agent may be written as Q⁺OH⁻, and will bind with the pH sensitive luminophore to form a Q⁺R⁻ ion-pair. This ion-pair benefits from the solubility of the phase transfer agent in hydrophobic solvents. The latter may in turn be used to dissolve different polymers. Preferably, the phase transfer agent is TMAH for its lesser susceptibility towards Hofmann elimination compared to TOAH, or other quaternary ammonium ions.

Examples of plasticiser include, without being limited to, Tributyl phosphate (TPB), dioctylphthalate, tris(ethyl-hexyl) phosphate, Highty PSD, Brij 35, Tween20, etyltrimethylammonium bromide or Melflux 2651F.

Upon fabrication of the CO₂-sensitive luminescent layer, luminophore, phase transfer agent, polymer and plasticiser are mixed in an organic solvent, such as ethanol/water, DMF, DMSO, then spread on the CO₂-permeable layer, with knife- or spin-coating. When the solvent dries, the polymer will form a solid polymer matrix entrapping the Q⁺R⁻ ion-pairs in its hydrophilic matrix.

The CO₂-sensitive luminescent layer may have a thickness ranging from 1 μm to 100 μm, preferably ranging from 10 μm to 15 μm. Advantageously a thin layer offers a good compromise between response time (the thinner the better) and brightness (the thicker the brighter).

The sealing layer may comprise an adhesive such as, for example, acrylic adhesive (e.g. copolymers of alkyl ester monomers with a functional monomer such as acrylic acid) or silicone-based adhesive (i.e. silicone resin/silicone polymer gum mixture). This advantageously ensure good contact between the skin and the sensor, preventing CO₂ leakage or unwanted shift of the sensor on the skin.

The sealing layer may have a thickness ranging from 2 μm to 50 μm, preferably ranging from 5 μm to 25 μm, more preferably 10 μm.

The CO₂-permeable polymer layer may comprise polytetrafluoroethylene (PTFE), poly (dimethylsiloxane) (PDMS), hyflon, or a mixture thereof. Advantageously, these materials allow CO₂ diffusion from skin to the CO₂-sensitive luminescent layer while ensuring a protection against humidity loss and acidic vapors or sweat poisoning.

The CO₂-permeable polymer layer may have a thickness ranging from 5 μm to 150 μm, preferably ranging from 10 μm to 100 μm, more preferably ranging from 10 μm to 50 μm.

The CO₂-impermeable polymer layer may comprise polyethylene terephthalate (PET), polyethylene naphthalate (PEN), polycarbonate, poly(methyl methacrylate) (PMMA), or a mixture thereof. These materials advantageously are impermeable to CO₂, i.e. they prevent the diffusion of CO₂ outside of the sensor. Additionally, they can present smooth surface finish resulting in an enhanced optical coupling of the patch assembly and the sensing head.

The CO₂-impermeable polymer layer may have a thickness ranging from 50 μm to 300 μm, preferably ranging from 100 μm to 200 μm.

The patch assembly may have a thickness ranging from 20 μm to 500 μm, preferably ranging from 100 μm to 350 μm, more preferably from 150 μm to 200 μm.

The patch assembly may have a diameter ranging from 5 mm to 50 mm, preferably from 10 mm to 30 mm. Advantageously, a small size will be better tolerated by the wearer.

The patch assembly may have a volume to surface ratio ranging from 100 μm to 200 μm for a response time of 10 min.

In a preferred configuration, the patch assembly may comprise:
a sealing layer comprising an adhesive;
a PDMS CO₂-permeable polymer layer;
a CO₂-sensitive luminescent layer comprising HPTS and Ru-dpp dispersed in a polymer matrix;
a PET CO₂-impermeable polymer layer embedding the CO₂-sensitive luminescent layer.

In an alternate preferred configuration, the patch assembly may comprise:
a sealing layer comprising an adhesive;
a PDMS CO₂-permeable polymer layer;
a CO₂-sensitive luminescent layer comprising:
a first pH sensitive layer comprising HPTS dispersed in HPMC;
a second non-pH sensitive layer comprising Ru-dpp dispersed in PAN;

a PET CO₂-impermeable polymer layer embedding the CO₂-sensitive luminescent layer.

The substrate may be made of polyethylene terephthalate (PET), polyethylene naphthalate (PEN), poly (methyl methacrylate) (PMMA), polycarbonate, glass, or optical fibers. In the case of glass substrate, the latter may be treated with concentrated HNO₃ or solution comprising 95% H₂SO₄ and 30% H₂O₂) with further wash in acetone, ethanol, methanol, and de-ionised water followed by dried nitrogen cleaning, to promote adhesion of the patch.

The LED may emit light in the UVA-Visible-NIR range, preferably the LED may emit light in the visible range.

In a specific configuration, the LED may emit light at a wavelength ranging from 380 nm to 500 nm, preferably ranging from 425 nm to 475 nm, more preferably at 450 nm.

In a preferred configuration, the excitation spectra of the luminophores match the emission spectrum of the LED.

The LED may be coupled with an optical filter, preferably shortpass filter, more preferably a 450 nm cutoff shortpass filter. Advantageously, this excitation filter blocks the excitation signal so as not to pollute the light re-emitted by the CO₂-sensitive luminescent layer, which will be subsequently long-passed.

The LED may have an emission area ranging from 0.1 mm² to 5 mm², preferably from 0.5 mm² to 2 mm². In a most preferred configuration, the LED has an emission area of 1 mm².

The sensing head may comprise at least two LEDs configured to emit light to excite the luminophores in the CO₂-sensitive luminescent layer. Advantageously, using two LEDs allow for a more homogeneous illumination of the CO₂-sensitive luminescent layer leading to a better response of the luminophores. Also, this allows for an excitation at two distinct wavelengths which would allow ratiometric measurements.

The photodiode may be coupled with an optical filter, preferably longpass filter, more preferably a 500 nm cutoff longpass filter. Advantageously, this emission filter blocks the excitation signal while letting through the light re-emitted by the CO₂-sensitive luminescent layer and allows the photodiode to be sensitive to the filtered, re-emitted wavelengths.

The housing and substrate may contain additional masking elements, which may be made of fully opaque materials, black-paint, or a combination thereof. Said elements are intended to block internal reflections such as Fresnel reflections in the substrate, and so as to avoid direct internal light paths between the LED and the photodiode. Advantageously, those blocking elements may help to keep the light rays close to the optical axis, so that they arrive at an incidence close to normal onto the filters, thus avoiding shifts in the cutoff wavelengths of the latter.

The sensing head may comprise additional lenses to focus the light emitted by the LED onto the patch assembly, and to focus the light emitted by the luminophores onto the photodiode. Advantageously, those lenses may help to keep the light rays close to the optical axis, so that they arrive at an incidence close to normal onto the filters, thus avoiding shifts in the cutoff wavelengths of the latter.

In a preferred configuration, the sensing head comprises:
a glass substrate;
a LED emitting light at 450 nm coupled with a 450 nm cutoff shortpass filter;
a photodiode coupled with a 500 nm cutoff longpass filter;
a housing totally encapsulating the substate, LED and photodiode.

The sensor may further comprise an amplifier configured to amplify the signal detected by the photodiode.

The sensor may further comprise an acquisition board configured to acquire the signal detected by the photodiode or amplified by the amplifier.

The patch assembly may be disposable, i.e. single use, while the sensing head may be reused for multiples patches.

Method for transcutaneous determination of arterial CO₂ partial pressure

The invention also relates to a method for transcutaneous determination of arterial CO₂ partial pressure using luminescence comprising:
providing a sensor according to any one of claims 1 to 13;
placing said sensor on skin of a subject;
exciting the luminophores in the CO₂-sensitive luminescent layer with the at least one light emitting diode;
detecting light emitted by said luminophores with the at least one photodiode;
comparing at least one luminescence property of the pH sensitive luminophore and the non-pH sensitive luminophore;
determining arterial CO₂ partial pressure based on said comparison.

The arterial CO₂ partial pressure may be determined using Förster Resonance Energy Transfer, dual-lifetime referencing, Inner Filter Effect, or ratiometric probing between the pH sensitive luminophore and the non-pH sensitive luminophore.

In a preferred configuration, the arterial CO₂ partial pressure is determined using Förster Resonance Energy Transfer (FRET). FRET is a distance-dependent interaction between two light-sensitive molecules (luminophores), a donor and an acceptor. It involves the non-radiative transfer of energy from the excited state of the donor luminophore to the acceptor luminophore, which then emits light. In this case, emission intensity of both luminophores is compared.

In a preferred configuration, the arterial CO₂ partial pressure is determined by time-based Dual-Lifetime Referencing (t-DLR). t-DLR is a technique used to measure the luminescence lifetimes of both luminophores. By comparing the lifetime of the pH-sensitive luminophore to that of the non-pH-sensitive luminophore, it is possible to accurately determine the CO₂ pressure. This comparison helps to compensate for any potential fluctuations in signal strength, environmental changes, or sensor movement, leading to more reliable measurements. In other words, when the LED emits light to excite both luminophores, the rise of luminescence intensity of the pH sensitive luminophore (IFLU) is almost instantaneous compared to that of the non-pH sensitive luminophore (IREF). Similarly, when the LED stops emitting light, the decay in luminescence intensity of the pH sensitive luminophore is also instantaneous, leaving only the slow decay of the non-pH sensitive luminophore. pCO₂ can then be directly calculated from the IFLU/IREF ratio.

In this configuration, the reference luminophore R is the non-pH sensitive luminophore and the pH-sensitive luminophore P, the pH modified form of the luminophore (typically anionic form) being noted hereafter P⁻, and they emit light at specific wavelengths. It can then be shown that:

$$[ {P}^{-} ] = - α \cdot ( cotan ( {φ}_{mes} ) + β \qquad ( 4 )$$

wherein α and β are defined as:

$$\begin{matrix}α = ω \cdot {τ}_{R} \cdot \frac{\sqrt{1 + {( ω \cdot {τ}_{P} )}^{2}}}{1 + {( ω \cdot {τ}_{R} )}^{2}} \cdot \frac{{ε}_{P , λ}}{{ε}_{R , λ}} \cdot \frac{{Φ}_{R}}{{Φ}_{P}} \cdot [ R ] \\ β = \frac{1}{ω \cdot {τ}_{R}}\end{matrix} \qquad ( 5 )$$

with ω the probing signals pulsation, ε_{X}, λ the molar extinction coefficient of the luminophore X at the measurement wavelength λ, τ_{X} its fluorescence lifetime, and Φ_{X} its quantum yield. Thus, measuring the phase shift φₘₑₛ of the collected signal gives an information on [P⁻], which is itself a function of the pCO₂ to which the sensor is exposed. Indeed, by choosing a long-living reference luminophore (R), and a short-living, pH-sensitive luminophore (P), the phase shift φₘₑₛ of the total re-emitted signal depends on the surrounding pH. The re-emitted signal of the reference luminophore (R) is insensitive to pH changes and always has the same amplitude, while the re-emitted signal of the pH-sensitive luminophore (P) is of higher amplitude at higher pH, thus dragging φₘₑₛ towards a lower value at high pH. At low pH, the opposite happens.

In a preferred configuration, the arterial CO₂ partial pressure is determined by frequency-based Dual-Lifetime Referencing (f-DLR). f-DLR is a technique used to measure the concentration ratio of both luminophores. By illuminating the two luminophores with a sinusoidal light excitation signal and measuring the phase shift between the re-emitted signal and the excitation signal, the ratio of the concentrations of the reference luminophore and the pH-sensitive fluorophore can be retrieved. From this ratio, it is possible to accurately determine the CO₂ pressure. This comparison helps to compensate for any potential fluctuations in signal strength, environmental changes, or sensor movement, leading to more reliable measurements.

During the excitation step, the light emitting diode may emit light at a wavelength corresponding to the absorption peak of both luminophores. Preferably, the LED may emit light at a wavelength ranging from 380 nm to 500 nm, preferably ranging from 425nm to 475 nm, more preferably at 450 nm.

During the excitation step, the excitation time may range from 5 ms to 50 ms. In the event of a poor signal-to-noise ratio, the excitation time can be increased to 200 ms.

The sensor may be placed on the torso, a side, a forearm, an arm or a wrist.

### Method for Fabricating the Sensor

The invention also relates to a method for fabricating the sensor described in this disclosure, said method comprising:
i. assembling the patch assembly of the sensor;
ii. assembling the sensing head of the sensor; and
iii. coupling the patch assembly with the sensing head.

Step (i) of the patch assembly comprises:
a) fabrication of the CO₂ impermeable layer;
b) deposition of a mixture comprising a pH sensitive luminophore and a non-pH sensitive luminophore, polymer matrix, optionally a plasticizer and optionally a phase transfer agent in a solvent;
c) evaporation of said solvent resulting in the formation of the CO₂-sensitive luminescent layer;
d) deposition of the CO₂-permeable polymer layer; and
e) deposition of the sealing layer.

Deposition of layers may be performed by drop casting, dip coating, knife coating, spraying, or conventional roll-to-roll processes.

Layers may they may hold in place thanks to an double-sided adhesive ring placed around the CO₂-sensitive luminescent layer in a concentric design.

Step (iii) may comprise using double sided adhesive between the path assembly and the sensing head, or positioning the sensing head on top of the patch assembly and securing it with either an adhesive stuck onto the skin, or using a strap—e.g. a wrist or armband for instance.

### BRIEF DESCRIPTION OF THE DRAWINGS

FIG. 1 is a sensor 1 according to a first embodiment.

FIG. 2 is a sensor 1 according to a second embodiment.

FIG. 3 is a sensor 1 according to a third embodiment.

FIG. 4 is a sensor 1 according to a fourth embodiment.

FIG. 5 is a graph illustrating the phase response of the sensor of example 2 (in black) upon exposure to different CO₂ levels (in grey). The dash-dotted, vertical lines correspond to the times at which the gaseous mixture in the enclosure was changed from pure N₂ to 5% CO₂, 10% CO₂, and pure N₂ again.

### ILLUSTRATIVE EMBODIMENTS OF THE INVENTION

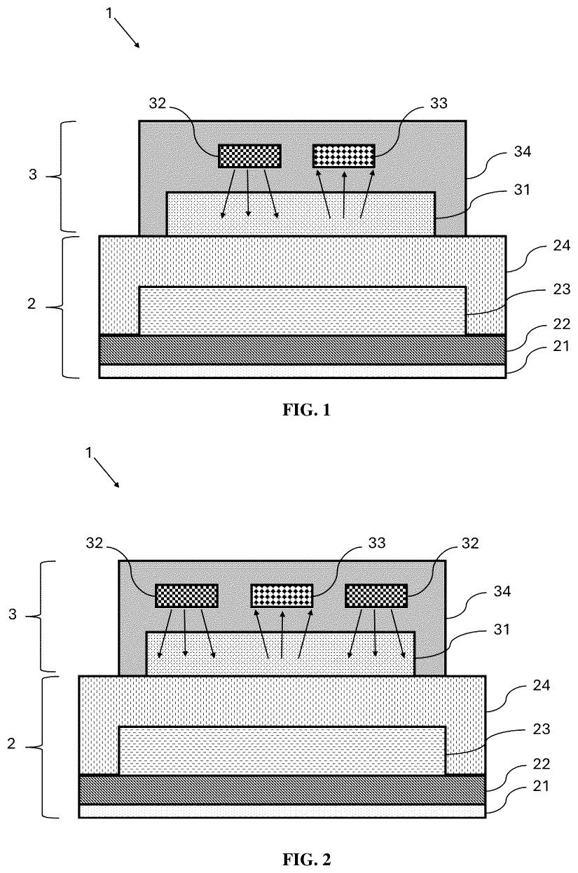

According to a first embodiment illustrated in FIG. 1, the sensor 1 comprises:
a patch assembly 2 comprising:
   a sealing layer 21 configured to ensure a contact between skin and the sensor 1;
   a CO₂-permeable polymer layer 22 configured to allow diffusion of CO₂ from the skin in the patch assembly 2;
   a CO₂-sensitive luminescent layer 23 comprising a pH sensitive luminophore and a non-pH sensitive luminophore;
   a CO₂-impermeable polymer layer 24 embedding the CO₂-sensitive luminescent layer 23;
   a sensing head 3 comprising:
   a substrate 31;
   a LED 32 configured to emit light (see descending arrows) to excite the luminophores in the CO₂-sensitive luminescent layer 23;
   a photodiode 33 configured to detect light emitted by the luminophores (see ascending arrows) in the CO₂-sensitive luminescent layer 23;
   a housing 34 totally encapsulating the substate 31, the LED 32 and the photodiode 33.

According to a second embodiment illustrated in FIG. 2, the sensor 1 comprises:
a patch assembly 2 comprising:
   a sealing layer 21 configured to ensure a contact between skin and the sensor 1;
   a CO₂-permeable polymer layer 22 configured to allow diffusion of CO₂ from the skin in the patch assembly 2;
   a CO₂-sensitive luminescent layer 23 comprising a pH sensitive luminophore and a non-pH sensitive luminophore;
   a CO₂-impermeable polymer layer 24 embedding the CO₂-sensitive luminescent layer 23;
   a sensing head 3 comprising:
   a substrate 31;
   two LEDs 32 configured to emit light (see descending arrows) to excite the luminophores in the CO₂-sensitive luminescent layer 23;
   a photodiode 33 configured to detect light emitted by the luminophores (see ascending arrows) in the CO₂-sensitive luminescent layer 23;
   a housing 34 totally encapsulating the substate 31, the LEDs 32 and the photodiode 33.

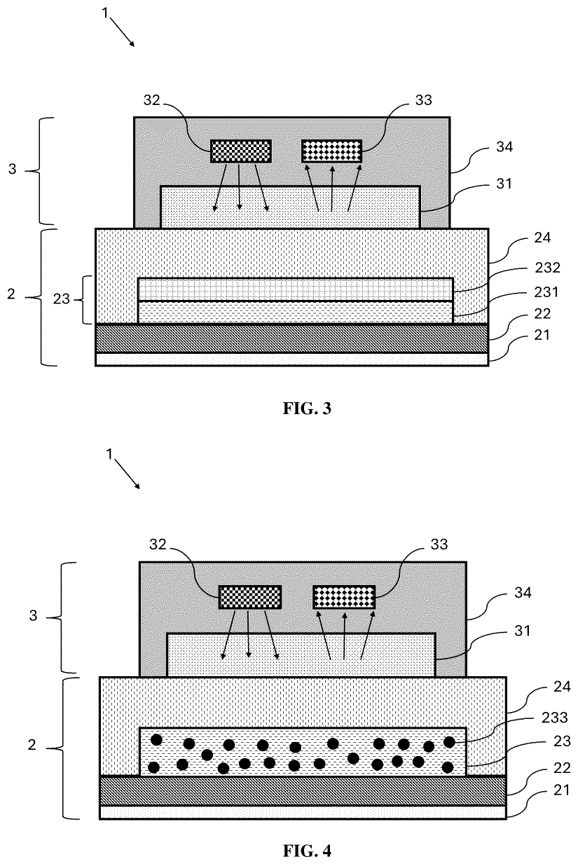

According to a third embodiment illustrated in FIG. 3, the sensor 1 comprises:
a patch assembly 2 comprising:
   a sealing layer 21 configured to ensure a contact between skin and the sensor 1;
   a CO₂-permeable polymer layer 22 configured to allow diffusion of CO₂ from the skin in the patch assembly 2;
   a CO₂-sensitive luminescent layer 23 comprising:
      a first pH sensitive layer 231 comprising the pH sensitive luminophore dispersed in a first polymer matrix;
      a second non-pH sensitive layer 232 comprising the non-pH sensitive luminophore dispersed in a second polymer matrix;
   a CO₂-impermeable polymer layer 24 embedding the CO₂-sensitive luminescent layer 23;
   a sensing head 3 comprising:
   a substrate 31;
   a LED 32 configured to emit light (see descending arrows) to excite the luminophores in the CO₂-sensitive luminescent layer 23;
   a photodiode 33 configured to detect light emitted by the luminophores (see ascending arrows) in the CO₂-sensitive luminescent layer 23;
   a housing 34 totally encapsulating the substate 31, the LED 32 and the photodiode 33.

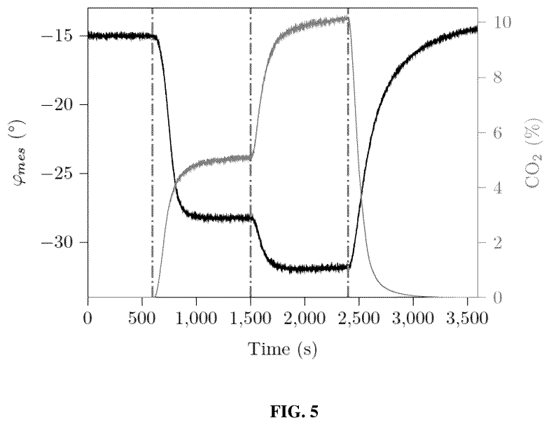

According to a fourth embodiment illustrated in FIG. 4, the sensor 1 comprises:
a patch assembly 2 comprising:
   a sealing layer 21 configured to ensure a contact between skin and the sensor 1;
   a CO₂-permeable polymer layer 22 configured to allow diffusion of CO₂ from the skin in the patch assembly 2;
   a CO₂-sensitive luminescent layer 23 comprising: the pH sensitive luminophore dispersed in a polymer matrix; and polymer beads 233 comprising the non-pH sensitive luminophore in a polymer;
   a CO₂-impermeable polymer layer 24 embedding the CO₂-sensitive luminescent layer 23;
   a sensing head 3 comprising:
   a substrate 31;
   a LED 32 configured to emit light (see descending arrows) to excite the luminophores in the CO₂-sensitive luminescent layer 23;
   a photodiode 33 configured to detect light emitted by the luminophores (see ascending arrows) in the CO₂-sensitive luminescent layer 23;
   a housing 34 totally encapsulating the substate 31, the LED 32 and the photodiode 33.

### EXAMPLES

The present invention is further illustrated by the following examples.

### Example 1: CO₂-sensitive luminescent layer fabrication

CO₂-sensitive luminescent layer was fabricated using 25×25 mm polished soda lime glass substrates, onto which two polymer layers were successively knife-coated on top of each other. The first layer consisted of a 100 μm-thick wet film of the following solution: 52.4 mg Ru-dpp and 524 mg polyacrylonitrile (PAN) in 5 mL dimethylformamide (DMF).

After complete solvent evaporation at room temperature, the second layer was applied, which consisted of a 100 μm-thick wet film of the following solution: 47.0 mg HPTS, 993 mg hydroxy propyl methylcellulose (HPMC) and 60 μL of a 25% tetraethyl ammonium hydroxide (TEAH) solution in methanol (˜1.5 M), in 10 mL of a 50:50 ethanol/distilled water mixture.

The total thickness of the so-obtained CO₂-sensitive luminescent layer was measured to be 10.9 μm using a surface profiler (Dektak 150, Bruker, USA).

### Example 2: in-Vitro Measurement

A sensor was provided. Said sensor comprised:
a patch assembly comprising:
   a sealing layer comprising an adhesive;
   a PDMS CO₂-permeable polymer layer;
   a CO₂-sensitive luminescent layer of example 1;
   a PET CO₂-impermeable polymer layer embedding the CO₂-sensitive luminescent layer;
   a sensing head comprising:
   a glass substrate;
   a LED emitting light at 450 nm coupled with a 450 nm cutoff shortpass filter;
   a photodiode coupled with a 500 nm cutoff longpass filter;
   a housing totally encapsulating the substate, LED and photodiode.

Two distinct measurement sessions were performed. At first, the sensors were submitted to water-saturated 0, 5, 10 and back to 0% CO₂ in N₂ at 25° C., while measuring φₘₑₛ every second. A 50 kHz excitation frequency was used with a 15 mA amplitude/20mA offset LED driving signal, while taking N=10 k samples per measurement at 250 kspl/s. Then, the CO₂ concentration was fixed to 5% while the sample count was varied from 50 to 50k with a 15 mA fixed excitation signal amplitude in a first set of measurements. In a second phase, the amplitude of the driving signal was varied from 1 up to 24 mA with a fixed N=10 k sample count and 24 mA offset. In these latter two configurations, 300 phase measurements were taken and their Root Mean Square Error (RMSE) was computed.

The excitation frequency and sampling rate were the same in all measurements. Most importantly, the excitation frequency fo, sampling frequency fs, and sample count N, were chosen such that N.fo/fs is an integer, in order to ensure a synchronous sampling situation. Finally, all experiments were performed under 100% relative humidity, consistently with expected levels under occluded skin.

FIG. 5 shows the sensor response to different CO₂ levels. Initially, in pure N₂, all the HPTS is present in its strongly fluorescent anionic form. Since the fluorescence time of HPTS is only of a few nanoseconds, as opposed to that of Ru-dpp which is in the microsecond range, the measured phase φₘₑₛ is maximal, being “lifted” upwards by HPTS. At the opposite, when the percentage of CO₂ inside the enclosure rises, the HPTS gradually turns into its non-fluorescing protonated form. Thus, φₘₑₛ shifts towards lower values, being “dragged” downward by the long fluorescence time of Ru-dpp.

### REFERENCES

1—sensor//2—patch assembly//21—sealing layer//22—CO₂-permeable polymer layer//23—CO₂-sensitive luminescent layer//231—first pH sensitive layer//232—second non-pH sensitive layer//233—polymer bead comprising non-pH sensitive luminophore//24—CO₂-impermeable polymer layer//3—sensing head//31—substrate//32—light emitting diode (LED)//33—photodiode//34—housing

1. A sensor for transcutaneous determination of arterial CO₂ partial pressure using luminescence, said sensor comprising:
a patch assembly comprising:
a sealing layer configured to ensure a contact between skin and the sensor;
a CO₂-permeable polymer layer configured to allow diffusion of CO₂ from the skin in the patch assembly;
a CO₂-sensitive luminescent layer comprising a pH sensitive luminophore and a non-pH sensitive luminophore;
a CO₂-impermeable polymer layer embedding the CO₂-sensitive luminescent layer;
a sensing head comprising:
a substrate;
at least one light emitting diode configured to emit light to excite the luminophores in the CO₂-sensitive luminescent layer; at least one photodiode configured to detect light emitted by the luminophores in the CO₂-sensitive luminescent layer;
a housing partially or totally encapsulating the substate, the at least one light emitting diode and the at least one photodiode;
wherein the arterial CO₂ partial pressure is determined by comparison of at least one luminescence property of the pH sensitive luminophore and the non-pH sensitive luminophore;
wherein the mass ratio between the pH sensitive luminophore and the non-pH sensitive luminophore in the CO₂-sensitive luminescent layer is ranging from 10% and 90%.

2. The sensor according to claim 1, wherein the loading charge of the pH sensitive luminophore in the CO₂ -sensitive luminescent layer is at least 0.5%, said loading charge being the mass ratio between the mass of luminophore in the CO₂-sensitive luminescent layer and the mass of said CO₂-sensitive luminescent layer.

3. The sensor according to claim 1, wherein the loading charge of the non-pH sensitive luminophore in the CO₂-sensitive luminescent layer is at least 0.5%, said loading charge being the mass ratio between the mass of luminophore in the CO₂-sensitive luminescent layer and the mass of said CO₂-sensitive luminescent layer.

4. The sensor according to claim 1, wherein the pH sensitive luminophore is selected among 1-hydroxy-pyrene-3,6,8-trisulfonate, (2′, 7′-Bis-(2-Carboxyethyl)-5-Carboxyfluorescein), seminaphtharhodafluorescein, 4,4-difluoro-5,7-dimethyl-4-bora-3a, 4a-diaza-s-indacene-3-propionic acid, other fluorescein derivatives, bromothymol blue, methyl red, litmus, cresol blue, bromocresol purple, chlorophenol red, phenol red, naphtolphtalein, phenolphthalein, cresolphtalein, nitrophenol, azolitmin, neutral red, rosolic acid, tropeolin OOO (1 and 2), or a mixture thereof.

5. The sensor according to claim 1, wherein non-pH sensitive luminophore is selected among tris(4,7-diphenyl-1,10 phenanthroline) ruthenium (II) dichloride (Ru-dpp), tris(1,10 phenanthroline) ruthenium (II) dichloride (Ru-pn), tris(2,2′-bipyridyl) ruthenium (II) dichlo-ride (Ru-bpy), pyrene, platinum octaethylporphyrin (ketone) (PtOEP (K)), palladium tetraphenyl tetrabenzoporphine (PdTPTBP), platinium tetraphenyl tetrabenzoporphine (PtTPTBP), Ir(Cn)₂(acac), Ir(Cs)₂(acac), cyclometalated iridium (III), coumarin complexes, or a mixture thereof.

6. The sensor according to claim 1, wherein the CO₂-sensitive luminescent layer comprises:
a first pH sensitive layer comprising the pH sensitive luminophore dispersed in a first polymer matrix;
a second non-pH sensitive layer comprising the non-pH sensitive luminophore dispersed in a second polymer matrix and being deposited on top of the first layer.

7. The sensor according to claim 1, wherein the CO₂-sensitive luminescent layer comprises a mixture of the pH sensitive luminophore and the non-pH sensitive luminophore in a polymer matrix.

8. The sensor according to claim 1, wherein the CO₂-sensitive luminescent layer comprises:
a polymer matrix;
the pH sensitive luminophore dispersed in the polymer matrix; and
polymer beads comprising the non-pH sensitive luminophore in a polymer, said polymer beads being dispersed in the polymer matrix.

9. The sensor according to claim 1, wherein the polymer matrix comprises ethyl cellulose, methyl cellulose, hydroxyethyl cellulose, hydroxymethyl cellulose, sodium carboxymethyl cellulose, hydroxyethyl methacrylate, hydroxypropyl methylcellulose, starches, gelatins, polyacrylonitrile (PAN), polytetrafluoroethylene (PTFE), polyethylene (PET), polystyrene (PS), poly(butylene adipate) polyester-based polyurethane, poly(methyl methacrylate) (PMMA), polyvinyl fluoride (PVF), poly(vinylidene chloride-co-vinyl chloride) (PVCD), BTDA-p,p′-DAS, poly(p-phenylene terephthalamide) (PPTA), poly(isobutyl methacrylate) (polyIBM), ethylene-vinyl alcohol copolymer (EVOH), polyvinyl alcohol (PVA), silicone, ormosil glass, or a mixture thereof.

10. The sensor according to claim 1, wherein the arterial CO₂ partial pressure is determined using Förster Resonance Energy Transfer, dual-lifetime referencing, Inner Filter Effect, or ratiometric probing between the pH sensitive luminophore and the non-pH sensitive luminophore.

11. The sensor according to claim 1, wherein the CO₂-sensitive luminescent layer has a thickness ranging from 1 μm to 100 μm.

12. The sensor according to claim 1, wherein the patch assembly has a thickness ranging from 20 μm to 500 μm.

13. The sensor according to claim 1, wherein the CO₂-permeable polymer layer comprises polytetrafluoroethylene, poly(dimethylsiloxane), hyflon, or a mixture thereof.

14. A method for transcutaneous determination of arterial CO₂ partial pressure using luminescence comprising:
providing a sensor according to claim 1;
placing said sensor on skin of a subject;
exciting the luminophores in the CO₂-sensitive luminescent layer with the at least one light emitting diode;
detecting light emitted by said luminophores with the at least one photodiode;
comparing at least one luminescence property of the pH sensitive luminophore and the non-pH sensitive luminophore;
determining arterial CO₂ partial pressure based on said comparison.

15. The method according to claim 14, wherein the arterial CO₂ partial pressure is determined using Förster Resonance Energy Transfer, dual-lifetime referencing, Inner Filter Effect, or ratiometric probing between the pH sensitive luminophore and the non-pH sensitive luminophore.

16. A method for fabricating a sensor according to claim 1, comprising:
assembling the patch assembly of the sensor;
assembling the sensing head of the sensor; and
coupling the patch assembly with the sensing head.
